(19) United States
(12) Patent Application Publication
(10) Pub. No.: US 2026/0021106 A1
(43) Pub. Date: Jan. 22, 2026

(54) COMBINATION THERAPY FOR TREATING CANCERS

(71) Applicant: GEISTLICH PHARMA AG, Wolhusen (CH)

(72) Inventors: James C. COSTIN, Phoenixville, PA (US); Hanns MOEHLER, Maennedorf (CH)

(73) Assignee: GEISTLICH PHARMA AG, Wolhusen (CH)

(21) Appl. No.: 18/995,420

(22) PCT Filed: Aug. 5, 2023
(86) PCT No.: PCT/IB2023/057934

§ 371 Date: Jan. 16, 2025

Related U.S. Application Data
(60) Provisional application No. 63/370,570, filed on Aug. 5, 2022.
(60) Provisional application No. 63/504,377, filed on May 25, 2023.

(51) Int. Cl. A61K 31/54; A61K 39/00; A61K 47/10; A61P 35/00; C07K 16/22

(52) U.S. Cl. CPC A61K 31/54; A61K 47/10; A61P 35/00; C07K 16/22; A61K 2039/505; A61K 2039/54; A61K 2039/545

(57) ABSTRACT

Methods, compounds and combinations useful for treating or delaying progression of cancer or reducing ascites formation or volume in an individual comprising administering to the individual an effective amount of an anti-VEGF drug and a compound of Formula I:, wherein R is H, an in vivo cleavable linker or group, or a leaving group in aqueous solution and R₁ and R₂ are independently, H, alkyl, an aryl, a substituted alkyl, a substituted phenyl, a substituted aryl, or a combination thereof.

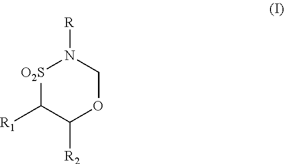

### FIELD OF THE DISCLOSURE

This disclosure relates to compositions, kits, and methods for treating, inhibiting, preventing or reducing cancers or reducing ascites formation or volume in a subject by administering combination therapies of the present disclosure.

### BACKGROUND

The failure of a significant number of cancer patients to respond to drug and/or radiation therapy is a serious problem in the treatment of cancer. Many forms of cancer resist effective intervention despite various advances in the field of oncology. The build-up of fluid in the peritoneal cavity—ascites—is a hallmark of ovarian cancer, the most lethal of all gynaecological malignancies. This remarkable fluid, which contains a variety of cellular and acellular components, is known to contribute to patient morbidity and mortality by facilitating metastasis and contributing to chemoresistance, but remains largely under-researched. As a comorbidity, ascites can have deleterious effects on a patient's quality of life, as it is commonly accompanied by dyspnoea, abdominal tenderness and pain, nausea, anorexia, fatigue and impaired movement. Ovarian cancer is the most lethal gynaecological malignancy, with more than 125,000 women dying from this disease every year worldwide. This figure has been predicted to rise by 67% to >250,000 women by the year 2035. The most common and aggressive subtype of ovarian cancer is high-grade serous ovarian cancer (HGSOC). Ascites is present in more than one third of ovarian cancer patients at initial diagnosis and in almost all cases of relapse. The greater the volume and frequency with which ascites accumulates in individual patients, the worse the prognosis. Although this poor prognosis is thought to be due to its tendency to present with HGSOC and in advanced stage disease (both independent predictors of poor prognosis), notably, ascites is known to contribute to chemoresistance, metastasis and decreased resectability.

Accordingly, there is a long-felt and unmet need for new compositions and methods to treat, inhibit, prevent or reduce cancers in a subject by administering combination therapies that increase the efficacy of cancer therapies. There is also a long-felt and unmet need for new compositions and methods to treat, inhibit, prevent or reduce ascites formation or ascites volume in a subject by administering combination therapies.

### SUMMARY OF THE INVENTION

In one aspect, the present disclosure includes a method of treating or delaying progression of cancer in an individual or reducing volume of ascites in the individual or reducing formation of ascites in the individual comprising administering to the individual an effective amount of an anti-VEGF drug and a compound of Formula I:

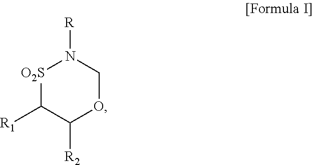

wherein R is H, an in vivo cleavable linker or group, or a leaving group in aqueous solution and R₁ and R₂ are independently, H, alkyl, an aryl, a substituted alkyl, a substituted phenyl, a substituted aryl, or a combination thereof, or a compound selected from the group consisting of the following:

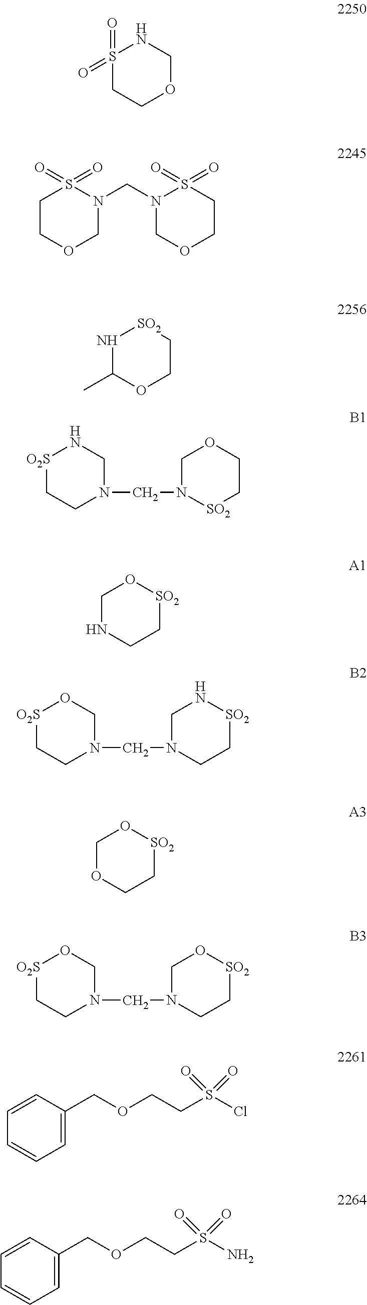

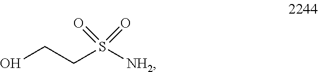

a pharmaceutically acceptable salt, hydrate, ester, prodrug, or solvate thereof. In some aspects, the substituted alkyl, substituted phenyl, or substituted aryl may be substituted with any appropriate molecule including, e.g., one or more halogens or halogen-containing molecules, one or more hydroxyl groups, one or more acyl groups, one or more acyloxy groups, one or more alkoxy groups, one or more aryl groups, one or more carboxy groups, one or more carbonyl groups, one or more alkylcarboxy groups, one or more alkylsufonoxy groups, one or more alkylcarbonyl groups, one or more nitro groups, one or more cyano groups, one or more acylamido groups, one or more phenyl groups, one or more tolyl groups, one or more chlorophenyl groups, one or more alkoxyphenyl groups, one or more halophenyl groups, one or more benzoxazole groups, one or more thiazoline groups, one or more benzimidazole groups, one or more oxazole groups, one or more thiazole groups, one or more indole groups, or a combination thereof.

In one aspect, the compound of Formula I is compound 2250 (also referred to as GP-2250).

In one aspect, the anti-VEGF drug is an anti-VEGFR2 antibody; an anti-VEGFR1 antibody; a VEGF-trap; a bispecific VEGF antibody; a bispecific antibody comprising a combination of two arms selected from the group consisting of an anti-VEGF arm, an anti-VEGFR1 arm, and an anti-VEGFR2 arm; an anti-VEGF-A antibody; an anti-VEGFB antibody; an anti-VEGFC antibody; an anti-VEGFD antibody; a nonpeptide small molecule VEGF antagonist; an anti-PDGFR inhibitor; and a native angiogenesis inhibitor.

In one aspect, the anti-VEGF drug comprises bevacizumab, ramucirumab, tanibirumab, aflibercept, icrucumab, ziv-aflibercept, MP-0250, vanucizumab, sevacizumab, VGX-100, pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, imatinib, tivozanib, or a combination thereof.

In one aspect, the compound of Formula I is in the form of a composition with a pharmaceutically acceptable carrier. In one aspect, the method or use comprises treating a subject suffering from cancer by administering a combination of a compound of formula I and bevacizumab. In one aspect, the method or use comprises treating a subject suffering from cancer by administering a combination of compound 2250 and bevacizumab.

In some aspects, the present disclosure includes a method of treating tumor stem cells in a subject by administering a combination of the present disclosure.

In some aspects, the present disclosure includes a method of administering the anti-VEGF drug via oral, intravenous, intraperitoneal, subcutaneous, intramuscular, topical, intradermal, intranasal or intrabronchial administration (for example as effected by inhalation). In some aspects, the present disclosure includes a method of administering the anti-VEGF drug via parenteral administration, e.g., intravenous administration.

In some aspects, the present disclosure includes a method further comprising administering the anti-VEGF drug and the compound of formula I in combination with 5-fluorouracil (5-FU)/leucovorin (LV)/irinotecan (IFL), 5-FU/LV/oxaliplatin (FOLFOX4), fluoropyrimidine, irinotecan, oxaliplatin, paclitaxel, PLD (pegylated liposomal doxorubicin), topotecan, cisplatin, Cisplatin/paclitaxel, topotecan/paclitaxel, carboplatin, carboplatin/paclitaxel (PC), gemcitabine, carboplatin/gemcitabine, interferon alfa (IFN), or a combination thereof.

Other features and characteristics of the subject matter of this disclosure, as well as the methods of operation, functions of related elements of structure and the combination of parts, and economies of manufacture, will become more apparent upon consideration of the following description, drawings, and the appended claims, all of which form a part of this specification.

### BRIEF DESCRIPTION OF THE DRAWINGS

FIG. 1: VEGF-induced Endothelial Sprouting: Impact of GP-2250, Spheroid assay. FIG. 1 shows a schematic endothelial tip cell (top). Tip cells form sprouts, and stalk cells form capillary lumen as shown in the image (bottom).

FIG. 2 shows sprouting of endothelial spheroids in human normal brain endothelial cells treated with PBS control, Delta-like 4 (DLL4), and GP-2250 (10 μg/ml).

FIG. 3 shows the effects of PBS control, Delta-like 4 (DLL4), and GP-2250 (10 μg/ml) on VEGF-induced endothelial cell sprouting. GP-2250 inhibits VEGF-induced endothelial cell sprouting.

FIG. 4 quantifies the effects of PBS control, Delta-like 4 (DLL4), and GP-2250 on VEGF-induced endothelial cell sprouting.

FIG. 5 schematically illustrates the experimental design for in vivo testing of the GP-2250 and bevacizumab on OVCAR8-L mice. The experiment includes testing: 1) IgG control (10 mice, 6.25 mg/kg, BW); 2) GP-2250 (10 mice, 500 mg/kg. 3 times per week); 3) Bevacizumab (10 mice, 6.25 mg/kg, BW) (“BEV”); and 4) the combination of GP-2250 and Bevacizumab.

FIG. 6 shows the body weight, tumor weight, and tumor nodule numbers of mice at take down (day 49) with mice in the control and treatment groups.

FIG. 7 shows the effect of GP-2250 on HIF1α and VEGF expression.

FIGS. 8A-8B show the cytotoxic effect of GP-2250 on ovarian cancer cells. FIG. 8A is a cell viability assay and FIG. 8B shows the IC50 of GP-2250.

FIG. 9 shows Western blots of ovarian cancer cells treated with GP-2250 for 24 hours following Western blotting.

FIG. 10A shows mRNA Hexokinase 1 and Hexokinase 2 expression profiles in the various cell lines. FIG. 10B shows the protein and activity of Hexokinase 1 and Hexokinase 2 in the various cell lines. FIG. 10C shows that GP-2250 inhibits hexokinase activity and protein expression levels. FIG. 10D shows siRNA targeting hexokinase 1 and 2 that GP-2250 inhibits activity and reduces cancer cell viability. (ns, not significant, **P<0.01; **P<0.001 (vs. control; Student t-test)).

FIGS. 11A-11B show pharmacodynamics studies of GP-2250. FIG. 11A shows a schematic of in vivo PD study of GP-2250. FIG. 11B shows a Western blot and AKT kinase assay of GP-2250.

FIGS. 12A-12C show the effect of GP-2250 alone or in combination with other anti-tumor agents. FIG. 12A shows a cell viability assay of GP-2250 alone, paclitaxel alone, or the combination of GP-2250 with paclitaxel. FIG. 12B shows a cell viability assay of GP-2250 alone, cisplatin alone, or the combination of GP-2250 with cisplatin. FIG. 12C shows a cell viability assay of GP-2250 alone, topotecan alone, or the combination of GP-2250 with topotecan. *P<0.05; **P<0.01; ***P<0.001 (Student t-test).

### DETAILED DESCRIPTION

While aspects of the subject matter of the present disclosure may be embodied in a variety of forms, the following description are merely intended to disclose some of these forms as specific examples of the subject matter encompassed by the present disclosure. Accordingly, the subject matter of this disclosure is not intended to be limited to the forms or aspects so described and illustrated.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific aspects of the invention, but their usage does not delimit the invention, except as outlined in the claims.

The terms “inhibiting,” “reducing,” or “prevention,” or any variation of these terms, when used in the claims and/or the specification includes any measurable decrease or complete inhibition to achieve a desired result.

Bevacizumab (Avastin®) is a recombinant humanized monoclonal IgG1 antibody that specifically binds to and blocks the biological effects of VEGF. Bevacizumab has been approved for treatment of the advanced stages of six common types of cancer: colorectal cancer, breast cancer, non-small cell lung cancer (NSCLC), ovarian cancer, cervical cancer, and kidney cancer, which collectively cause over 2.5 million deaths each year. In the United States, bevacizumab was the first anti-angiogenesis therapy approved by the FDA, and it is now approved for the treatment of at least six tumor types, e.g., colorectal cancer, NSCLC, brain cancer (glioblastoma), kidney cancer (renal cell carcinoma), ovarian cancer, and cervical cancer, and development of bevacizumab's use for treatment of multiple other cancer types is in progress.

Bevacizumab has shown promise as a co-therapeutic, demonstrating efficacy when combined with a broad range of chemotherapies and other anti-cancer treatments. For example, phase-III studies have demonstrated the beneficial effects of combining bevacizumab with standard chemotherapeutic regimens (see, e.g., Saltz et al., 2008, J. Clin. Oncol., 26:2013-2019; Yang et al., 2008, Clin. Cancer Res., 14:5893-5899; Hurwitz et al., 2004, N. Engl. J. Med., 350:2335-2342). However, as in previous studies of angiogenesis inhibitors, some of these phase-III studies have shown that a portion of patients experience incomplete response to the addition of bevacizumab to their chemotherapeutic regimens. Accordingly, there is a need for methods of identifying those patients that are likely to respond or have an improved response to not only angiogenesis inhibitors (e.g., bevacizumab) alone, but also combination therapies comprising angiogenesis inhibitors (e.g., bevacizumab).

In one aspect, the present disclosure includes a method of treating or delaying progression of cancer in an individual or reducing volume of ascites in the individual or reducing formation of ascites in the individual comprising administering to the individual an effective amount of an anti-VEGF drug and a compound of Formula I:

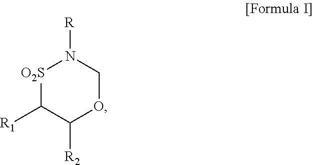

wherein R is H, an in vivo cleavable linker or group, or a leaving group in aqueous solution and R₂ and R₂ are independently, H, alkyl, an aryl, a substituted alkyl, a substituted phenyl, a substituted aryl, or a combination thereof, or a compound selected from the group consisting of the following:

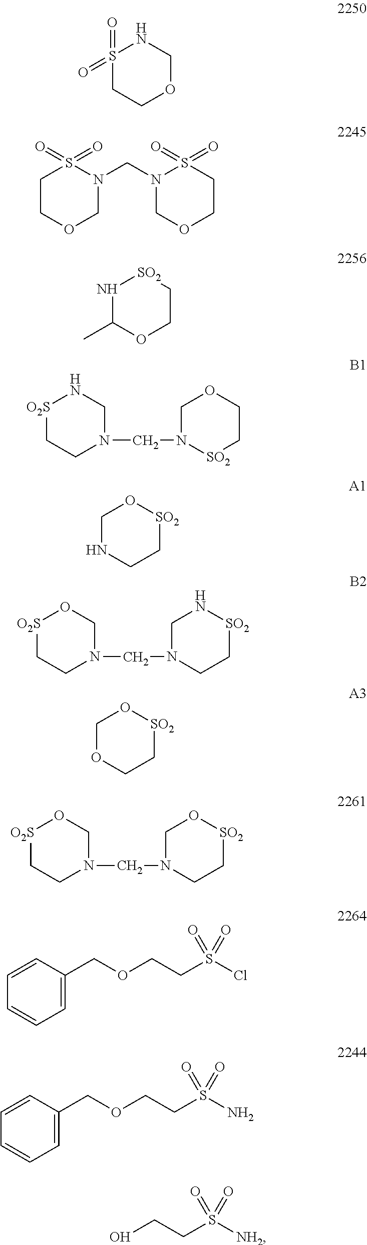

a pharmaceutically acceptable salt, hydrate, ester, prodrug, or solvate thereof. In some aspects, the substituted alkyl, substituted phenyl, or substituted aryl may be substituted with any appropriate molecule including, e.g., one or more halogens or halogen-containing molecules, one or more hydroxyl groups, one or more acyl groups, one or more acyloxy groups, one or more alkoxy groups, one or more aryl groups, one or more carboxy groups, one or more carbonyl groups, one or more alkylcarboxy groups, one or more alkylsufonoxy groups, one or more alkylcarbonyl groups, one or more nitro groups, one or more cyano groups, one or more acylamido groups, one or more phenyl groups, one or more tolyl groups, one or more chlorophenyl groups, one or more alkoxyphenyl groups, one or more halophenyl groups, one or more benzoxazole groups, one or more thiazoline groups, one or more benzimidazole groups, one or more oxazole groups, one or more thiazole groups, one or more indole groups, or a combination thereof.

In one aspect, the compound of Formula I is compound 2250 (also referred to as GP-2250).

In one aspect, the anti-VEGF drug is an anti-VEGFR2 antibody; an anti-VEGFR1 antibody; a VEGF-trap; a bispecific VEGF antibody; a bispecific antibody comprising a combination of two arms selected from the group consisting of an anti-VEGF arm, an anti-VEGFR1 arm, and an anti-VEGFR2 arm; an anti-VEGF-A antibody; an anti-VEGFB antibody; an anti-VEGFC antibody; an anti-VEGFD antibody; a nonpeptide small molecule VEGF antagonist; an anti-PDGFR inhibitor; and a native angiogenesis inhibitor.

In one aspect, the anti-VEGF drug comprises bevacizumab, ramucirumab, tanibirumab, aflibercept, icrucumab, ziv-aflibercept, MP-0250, vanucizumab, sevacizumab, VGX-100, pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, imatinib, tivozanib, or a combination thereof.

In one aspect, the compound of Formula I is in the form of a composition with a pharmaceutically acceptable carrier. In one aspect, the compound of Formula I is in the form of an orally administrable composition. In one aspect, the composition is in the form of a capsule, a tablet, or a pharmaceutically acceptable solution. In one aspect, the composition comprises the compound of Formula I at a concentration of about 0.01 to about 3% w/v. In one aspect, the composition comprises the compound of Formula I at a concentration of about 0.01 to about 1000 ug/ml. In one aspect, the composition contains one or more solubilizing agents. In one aspect, the composition comprises a polyol. In one aspect, the composition is an injection and/or infusion formulation comprising a pharmaceutically acceptable injection or infusion carrier.

In one aspect, the method or use comprises treating a subject suffering from cancer by administering a combination of a compound of formula I and bevacizumab. In one aspect, the method or use comprises treating a subject suffering from cancer by administering a combination of compound 2250 and bevacizumab.

In one aspect, the cancer is glioblastoma, glioma, neuroblastoma, astrocytoma, carcinomatous meningitis, colon cancer, rectal cancer, colorectal cancer, endometrial cancer, ovarian cancer, breast cancer, prostate cancer, lung cancer, mesothelioma, melanoma, renal cancer, liver cancer, pancreatic cancer, gastric cancer, esophageal cancer, urinary bladder cancer, cervical cancer, cardiac cancer, gall bladder cancer, skin cancer, bone cancer, cancers of the head and neck, leukemia, lymphoma, lymphosarcoma, adenocarcinoma, fibrosarcoma, or a metastasis thereof.

In some aspects, the cancer is biliary tract cancer; brain cancer, including glioblastomas and medulloblastomas; breast cancer; triple negative breast cancer; uterine cancer; tubal cancer; cervical cancer; choriocarcinoma; colon cancer; bladder cancer; endometrial cancer; retinoblastoma; vaginal cancer; vulvar cancer; esophageal cancer; mouth cancer; gastric cancer; kidney cancer; hematological neoplasms, including acute lymphocytic and myelogenous leukemia; multiple myeloma; AIDS-associated leukemias and adult T-cell leukemia lymphoma; intraepithelial neoplasms, including Bowen's disease and Paget's disease; liver cancer (hepatocarcinoma); lung cancer; head or neck cancers or oral cancers (mouth, throat, esophageal, nasopharyngeal, jaw, tonsil, nasal, lip, salivary gland, tongue, etc.); lymphomas, including Hodgkin's disease and lymphocytic lymphomas; neuroblastomas; neuroendocrine tumors; oral cancer, including squamous cell carcinoma; adrenal cancer; anal cancer; angiosarcoma; appendix cancer; bile duct cancer; bone cancer; carcinoid tumors; soft tissue sarcoma; rhabdomyosarcoma; eye cancer; ovarian cancer, including those arising from epithelial cells, stromal cells, germ cells and mesenchymal cells, and fallopian tube cancer; gallbladder cancer; pancreatic cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma[teratomas, choriocarcinomas]), stromal tumors and germ cell tumors; penile cancer; hemangioendothelioma; gastrointestinal cancer; ureteral cancer; urethral cancer; spinal cancer; pituitary gland cancer; primary central nervous system (CNS) lymphoma; thyroid cancer, including thyroid adenocarcinoma and medullar carcinoma; and renal cancer including adenocarcinoma and Wilms tumor. In some aspects, cancers or tumors include breast cancer, prostate cancer, colorectal cancer, lymphoma, multiple myeloma, and melanoma.

In some aspects, the present disclosure includes a method of treating tumor stem cells in a subject by administering a combination of the present disclosure.

In some aspects, the present disclosure includes a method of administering a dosage of 0.1-1,000 mg/kg of a compound of formula I in combination with 1 mg/kg to 100 mg/kg of the anti-VEGF drug. In some aspects, the present disclosure includes a method of administering a total daily dose of about 0.1 g to about 100 g of a compound of formula I.

In one aspect, the anti-VEGF drug (e.g., bevacizumab) is administered at 5 mg/kg of body weight given once every 2 weeks, 10 mg/kg of body weight given once every 2 weeks, 7.5 mg/kg of body weight given once every 3 weeks, or 15 mg/kg of body weight given once every 3 weeks.

In some aspects, the present disclosure includes a method of administering about 20 mg to about 2000 mg of the anti-VEGF drug. For example, the fixed dose may be approximately 420 mg, approximately 525 mg, approximately 840 mg, or approximately 1050 mg of the anti-VEGF drug. Where a series of doses are administered, these may, for example, be administered approximately every week, approximately every 2 weeks, approximately every 3 weeks, or approximately every 4 weeks, but preferably approximately every 3 weeks. The fixed doses may, for example, continue to be administered until disease progression, adverse event, or other time as determined by the physician. For example, from about two, three, or four, up to about 17 or more fixed doses may be administered. In some aspects, the stated dosages are reduced by 25-75% due to the synergistic effects of the combination of the anti-VEGF drug with the compound of formula I.

In some aspects, the present disclosure includes a method of administering the anti-VEGF drug via oral, intravenous, intraperitoneal, subcutaneous, intramuscular, topical, intradermal, intranasal or intrabronchial administration (for example as effected by inhalation). In some aspects, the present disclosure includes a method of administering the anti-VEGF drug via parenteral administration, e.g., intravenous administration.

In some aspects, the present disclosure includes a method of administering one or more loading dose(s) of the anti-VEGF drug (e.g., an anti-VEGF antibody such as bevacizumab) followed by one or more maintenance dose(s). A “loading” dose herein generally comprises an initial dose of a therapeutic agent administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s). A “maintenance” dose or “extended” dose herein refers to one or more doses of a therapeutic agent administered to the patient over a treatment period. Usually, the maintenance doses are administered at spaced treatment intervals, such as approximately every week, approximately every 2 weeks, approximately every 3 weeks, or approximately every 4 weeks. In another embodiment, a plurality of the same dose is administered to the patient. According to one preferred embodiment of the invention, a fixed dose of the anti-VEGF drug of approximately 840 mg (loading dose) is administered, followed by one or more doses of approximately 420 mg (maintenance dose(s)) of the antagonist. The maintenance doses are preferably administered about every 3 weeks, for a total of at least two doses, up to 17 or more doses. In some aspects, the stated dosages are reduced by 25-75% due to the synergistic effects of the combination of the anti-VEGF drug with the compound of formula I.

In some aspects, the present disclosure includes a method of administering one or more fixed dose(s) of approximately 1050 mg of the anti-VEGF drug, for example every 3 weeks. According to this embodiment, one, two or more of the fixed doses are administered, e.g., for up to one year (17 cycles), and longer as desired. In some aspects, the stated dosages are reduced by 25-75% due to the synergistic effects of the combination of the anti-VEGF drug with the compound of formula I.

In some aspects, the present disclosure includes a method of administering a fixed dose of approximately 1050 mg of the anti-VEGF drug as a loading dose, followed by one or more maintenance dose(s) of approximately 525 mg. About one, two, or more maintenance doses may be administered to the patient every 3 weeks according to this embodiment. In some aspects, the stated dosages are reduced by 25-75% due to the synergistic effects of the combination of the anti-VEGF drug with the compound of formula I.

As used herein, the terms “substantially” and “substantial” refer to a considerable degree or extent. When used in conjunction with, for example, an event, circumstance, characteristic, or property, the terms can refer to instances in which the event, circumstance, characteristic, or property occurs precisely as well as instances in which the event, circumstance, characteristic, or property occurs to a close approximation, such as accounting for typical tolerance levels or variability of the examples described herein.

As used herein, the term “about” is used to provide flexibility to a numerical range endpoint by providing that a given value may be “a little above” or “a little below” the endpoint. The degree of flexibility of this term can be dictated by the particular variable and would be within the knowledge of those skilled in the art to determine based on experience and the associated description herein. For example, in one aspect, the degree of flexibility can be within about ±10% of the numerical value. In another aspect, the degree of flexibility can be within about ±5% of the numerical value. In a further aspect, the degree of flexibility can be within about ±2%, ±1%, or ±0.05%, of the numerical value.

Generally herein, the term “or” includes “and” and “and/or.”

As used herein, a plurality of compounds or steps may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

The compounds of the invention may be useful in a free acid form, a free base form, in the form of pharmaceutically acceptable salts, pharmaceutically acceptable hydrates, pharmaceutically acceptable esters, pharmaceutically acceptable solvates, pharmaceutically acceptable prodrugs, pharmaceutically acceptable metabolites, and in the form of pharmaceutically acceptable stereoisomers. These forms are all within the scope of the invention. In practice, the use of these forms amounts to use of the neutral compound.

“Pharmaceutically acceptable salt”, “hydrate”, “ester” or “solvate” refers to a salt, hydrate, ester, or solvate of the inventive compounds which possesses the desired pharmacological activity and which is neither biologically nor otherwise undesirable. Organic acids can be used to produce salts, hydrates, esters, or solvates such as acetate, adipate, alginate, aspartate, benzoate, benzenesulfonate, p-toluenesulfonate, bisulfate, sulfamate, sulfate, naphthylate, butyrate, citrate, camphorate, camphorsulfonate, cyclopentanepropionate, digluconate, dodecylsulfate, ethanesulfonate, fumarate, glucoheptanoate, glycerophosphate, hemisulfate heptanoate, hexanoate, 2-hydroxyethanesulfonate, lactate, maleate, methanesulfonate, 2-naphthalenesulfonate, nicotinate, oxalate, tosylate and undecanoate. Inorganic acids can be used to produce salts, hydrates, esters, or solvates such as hydrochloride, hydrobromide, hydroiodide, and thiocyanate. Other pharmaceutically acceptable salts include, but are not limited to, hydrochloride, hydrobromide, sulphate, phosphate, tartrate, fumarate, maleate, oxalate, acetate, propionate, succinate, mandelate, mesylate, besylate and tosylate.

Salts, hydrates, esters, or solvates may also be formed with organic bases. Pharmaceutically acceptable base addition salts of acidic compounds may be formed with organic and inorganic bases by conventional methods. For example, alkali metal and alkaline earth metal hydroxides, carbonates and bicarbonates such as sodium hydroxide, potassium hydroxide, calcium hydroxide, potassium carbonate, sodium bicarbonate, magnesium carbonate and the like, ammonia, primary, secondary and tertiary amines and the like. Also aluminum salts of the instant compounds may be obtained by treating the corresponding sodium salt with an appropriate aluminum complex such as, for example, aluminum chloride hexahydrate, and the like. Non-toxic organic bases include, but are not limited to, triethylamine, butylamine, piperazine, and tri(hydroxymethyl)-methylamine. Examples of suitable base salts, hydrates, esters, or solvates include hydroxides, carbonates, and bicarbonates of ammonia, alkali metal salts such as sodium, lithium and potassium salts, alkaline earth metal salts such as calcium and magnesium salts, aluminum salts, and zinc salts. Organic bases suitable for the formation of pharmaceutically acceptable base addition salts, hydrates, esters, or solvates of the compounds of the present invention include those that are non-toxic and strong enough to form such salts, hydrates, esters, or solvates. For purposes of illustration, the class of such organic bases may include mono-, di-, and trialkylamines, such as methylamine, dimethylamine, triethylamine and dicyclohexylamine; mono-, di- or trihydroxyalkylamines, such as mono-, di-, and triethanolamine; amino acids, such as arginine and lysine; guanidine; N-methyl-glucosamine; N-methyl-glucamine; L-glutamine; N-methyl-piperazine; morpholine; ethylenediamine; N-benzyl-phenethylamine; (trihydroxy-methyl)aminoethane; and the like. See, for example, “Pharmaceutical Salts,” J. Pharm. Sci., 66:1, 1-19 (1977). Accordingly, basic nitrogen-containing groups can be quaternized with agents including: lower alkyl halides such as methyl, ethyl, propyl, and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as benzyl and phenethyl bromides.

The salts, hydrates, esters, or solvates of the basic compounds may be prepared either by dissolving the free base of a oxathiazin-like compound in an aqueous or an, aqueous alcohol solution or other suitable solvent containing the appropriate acid or base, and isolating the salt by evaporating the solution. Alternatively, the free base of the oxathiazin-like compound may be reacted with an acid, as well as reacting the oxathiazin-like compound having an acid group thereon with a base, such that the reactions are in an organic solvent, in which case the salt separates directly or can be obtained by concentrating the solution.

“Pharmaceutically acceptable prodrug” refers to a derivative of the inventive compounds which undergoes biotransformation prior to exhibiting its pharmacological effect(s). The prodrug is formulated with the objective(s) of improved chemical stability, improved patient acceptance and compliance, improved bioavailability, prolonged duration of action, improved organ selectivity, improved formulation (e.g., increased hydrosolubility), and/or decreased side effects (e.g., toxicity). The prodrug can be readily prepared from the inventive compounds using methods known in the art, such as those described by Burger's Medicinal Chemistry and Drug Chemistry, Fifth Ed., Vol. 1, pp. 172-178, 949-982 (1995). For example, the inventive compounds can be transformed into prodrugs by converting one or more of the hydroxy or carboxy groups into esters. Further, N-protected versions of the inventive compounds are also included as non-limiting examples of pharmaceutically acceptable prodrugs of the inventive compounds.

“Pharmaceutically acceptable metabolite” refers to drugs that have undergone a metabolic transformation. After entry into the body, most drugs are substrates for chemical reactions that may change their physical properties and biologic effects. These metabolic conversions, which usually affect the polarity of the compound, alter the way in which drugs are distributed in and excreted from the body. However, in some cases, metabolism of a drug is required for therapeutic effect. For example, anticancer drugs of the antimetabolite class must be converted to their active forms after they have been transported into a cancer cell. Since must drugs undergo metabolic transformation of some kind, the biochemical reactions that play a role in drug metabolism may be numerous and diverse. The main site of drug metabolism is the liver, although other tissues may also participate.

Furthermore, certain compositions, concentrations, dosage regimens, dosage amounts, syndromes or conditions, steps, or the like may be discussed in the context of one specific aspect. It is understood that this is merely for convenience, and such disclosure is equally applicable to other aspects found herein. For example, a list of method steps, active agents, kits or compositions described with respect to a method of administering a compound of the present disclosure would find direct support for aspects related to method steps, active agents, kits or compositions of, e.g., the following: treating, preventing, inhibiting or reducing at least one sign or symptom of a disease, disorder or condition of the present disclosure; treating, preventing, inhibiting or reducing at least one side effect of a drug administered to a subject suffering from a disease, disorder or condition of the present disclosure; treating, preventing, inhibiting or reducing the incidence of a sign or symptom of a disease, disorder or condition of the present disclosure, even if those method steps, active agents, kits or compositions are not re-listed in the context of that aspect in the specification.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilizing (i.e. not worsening) the state of disease, delaying or slowing of disease progression, amelioration or palliation of the disease state, diminishment of the reoccurrence of disease, and remission (whether partial or total), whether detectable or undetectable. “Treating” and “treatment” can also mean prolonging survival as compared to expected survival if not receiving treatment. In addition to being useful as methods of treatment, the methods described herein may be useful for the prevention or prophylaxis of disease. As used herein, the term “treating” may refer to any administration of a compound of the present invention and includes: (i) preventing or inhibiting the disease in a mammal, e.g., a human, that is experiencing or displaying the pathology or symptomatology of the diseased (i.e., arresting further development of the pathology and/or symptomatology); or (ii) ameliorating the disease in a mammal, e.g., a human that is experiencing or displaying the pathology or symptomatology of the disease (i.e., reversing the pathology and/or symptomatology). The term “controlling” includes preventing, treating, eradicating, ameliorating or otherwise reducing the severity of the condition being controlled.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 0.01 to 2.0” should be interpreted to include not only the explicitly recited values of about 0.01 to about 2.0, but also include individual values and sub-ranges within the indicated range. Thus, included in this numerical range are individual values such as 0.5, 0.7, and 1.5, and sub-ranges such as from 0.5 to 1.7, 0.7 to 1.5, and from 1.0 to 1.5, etc. Furthermore, such an interpretation should apply regardless of the breadth of the range or the characteristics being described. Additionally, it is noted that all percentages are in weight, unless specified otherwise.

In understanding the scope of the present disclosure, the terms “including” or “comprising” and their derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. It is understood that reference to any one of these transition terms (i.e. “comprising,” “consisting,” or “consisting essentially”) provides direct support for replacement to any of the other transition term not specifically used. For example, amending a term from “comprising” to “consisting essentially of” would find direct support due to this definition.

Some oxathiazin-like compounds are described in PCT/IB2015/059741, filed Dec. 17, 2015, which is incorporated herein by reference in its entirety. In certain aspects, oxathiazin-like compounds according to Formula I are utilized according to the invention wherein R is H, an in vivo cleavable linker or group, or a leaving group in aqueous solution, and R₁ and R₂ are independently, H, alkyl, an aryl, a substituted alkyl, a substituted phenyl, a substituted aryl, or a combination thereof. In some aspects, the substituted alkyl, substituted phenyl, or substituted aryl may be substituted with any appropriate molecule including, e.g., one or more halogens or halogen-containing molecules, one or more hydroxyl groups, one or more acyl groups, one or more acyloxy groups, one or more alkoxy groups, one or more aryl groups, one or more carboxy groups, one or more carbonyl groups, one or more alkylcarboxy groups, one or more alkylsufonoxy groups, one or more alkylcarbonyl groups, one or more nitro groups, one or more cyano groups, one or more acylamido groups, one or more phenyl groups, one or more tolyl groups, one or more chlorophenyl groups, one or more alkoxyphenyl groups, one or more halophenyl groups, one or more benzoxazole groups, one or more thiazoline groups, one or more benzimidazole groups, one or more oxazole groups, one or more thiazole groups, one or more indole groups, etc., or a combination thereof. In some aspects, the alkyl or substituted alkyl may be a C1 to C30 alkyl. In some aspects, the alkyl may be branched or unbranched. In some aspects, the aryl may be heterocyclic, polycyclic, or monocyclic.

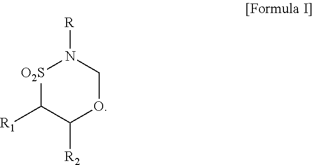

Exemplary oxathiazin-like compounds include the following:

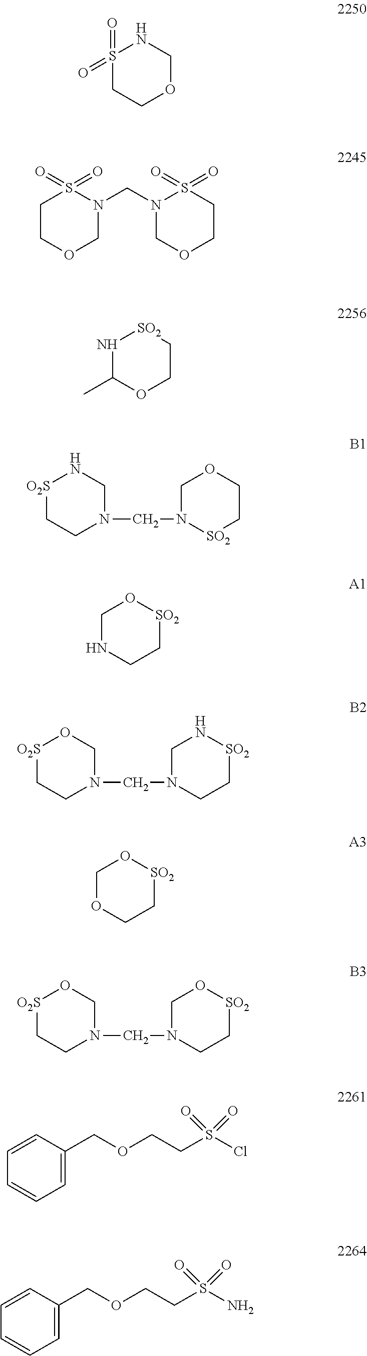

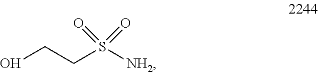

and isethionic acid hydroxymethylamide.

In certain aspects, the invention also relates to compositions, e.g., pharmaceutical compositions, containing the compounds, complexes, or conjugates described herein, including pharmaceutically acceptable solutions thereof, as well as administrable compositions, kits, medical devices, and pharmaceutical containers containing the compositions of the present disclosure.

The terms “effective amount” or “therapeutically effective amount” described herein means the amount of the subject compound that will elicit the biological or medical response of a tissue, system, animal or human that is being sought by the researcher, veterinarian, medical doctor or other clinician. In one example, the therapeutically effective amount comprises about 0.0001 to about 10,000 mg/kg, about 0.001 mg/kg to about 5,000 mg/kg, about 0.01 mg/kg to about 1,000mg/kg, about 0.05 mg/kg to about 750 mg/kg, about 0.1 mg/kg to about 600 mg/kg, about 1 mg/kg to about 500 mg/kg, about 10 mg/kg to about 400 mg/kg, about 20 mg/kg to about 300 mg/kg, about 200 mg/kg to about 500 mg/kg, about 300 mg/kg to about 400 mg/kg, about 250 mg/kg, 300 mg/kg, 400 mg/kg, 420 mg/kg, 450 mg/kg, about 500 mg/kg, or an dosage amount or range within any of the disclosed ranges of body weight of the subject.

The terms “administration of” or “administering a” compound as used herein should be understood to mean providing a compound of the invention to the individual in need of treatment in a form that can be introduced into that individual's body, e.g., intravenously, subcutaneously, intramuscularly, topically, orally, intraperitoneally, ophthalmically, by intravitreal injection, intrathecally, intranasally, intrapulmonary, transdermally, intraocularly, by inhalation, transtracheally, intravitreally, or a combination thereof. In some aspects, a compound of the invention may be administered in a therapeutically useful form and therapeutically useful amount, including, but not limited to: oral dosage forms, such as tablets, capsules, syrups, suspensions, and the like; injectable dosage forms, such as intravenous (IV), intramuscular (IM), or intraperitoneal (IP), intranasal, and the like; enteral or parenteral, transdermal dosage forms, including creams, jellies, powders, or patches; buccal dosage forms; inhalation powders, sprays, suspensions, and the like; and rectal suppositories.

Depending upon the particular route of administration desired a variety of pharmaceutically acceptable carriers well known in the art may be used. These include solid or liquid fillers, diluents, hydrotropes, surface-active agents, and encapsulating substances. Optional pharmaceutically active materials may be included, which do not substantially interfere with the activity of the one or more oxathiazin-like compounds.

As used herein the term “intravenous administration” includes injection, infusion, and other modes of intravenous administration.

The term “pharmaceutically acceptable” as used herein to describe a carrier, diluent or excipient must be compatible with the other ingredients of the formulation and not deleterious to the recipient thereof.

In one aspect, the present disclosure includes administering one or more compounds of the present disclosure alone or in combination with at least one second active agent. For example, in some aspects, the present disclosure includes administering one or more compounds of the present disclosure with an anti-angiogenesis agent, anti-autoimmune agent, and/or anti-neoplastic agent to a subject in need thereof.

In some aspects, the present disclosure includes administering one or more compounds and combinations of the present disclosure to a subject in need thereof to regulate mitochondrial function and protein production to reduce, inhibit, prevent and/or eliminate cancer stem cells (CSCs). In some aspects, the present disclosure includes administering one or more compounds and combinations of the present disclosure to a subject in need thereof to increase production or localization of reactive species, e.g., reactive oxygen species, in tumors and cancerous cells, thereby reducing cancer cell viability without affecting normal cells. In some aspects, the present disclosure includes administering one or more compounds and compositions of the present disclosure to a subject in need thereof to induce reversion of desmoplastic tissue surrounding cancer cells/tumors to normal extracellular matrix.

In some aspects, the present disclosure includes methods and compositions for treating a subject having cancer, autoimmune disease, angiogenesis or other disease, disorder, condition or symptom disclosed herein, comprising selecting a subject having cancer, autoimmune disease, angiogenesis or other disease, disorder, condition or symptom disclosed herein.

Non-limiting examples of such diseases, disorders and conditions include one or more of tumors, cancers including, but not limited to carcinoma, leukemia, lymphoma, melanoma, myeloma, sarcoma, a metastatic solid tumor, and mixed-type cancers, skin diseases (including, but are not limited to, psoriasis, telangiectasia, wound granularization, scleroderma, neovascularization as a consequence of infection (e.g., cat scratch disease, bacterial ulceration, etc.)), macular degeneration or age-related blindness, diabetic ulcers, chronic ulcers and wounds, stroke, traumatic brain injury, neovascularization of the retina, neovascularization of the cornea (such as that caused by trachoma, infections, inflammation, transplantations or trauma), diabetic retinopathy, diabetic retinal edema, diabetic macula edema, ischemic retinopathy, hypertensive retinopathy, occlusive retinopathy, retinopathy of prematurity, neovascularization subsequent to trauma, neovascularization subsequent to infection, neovascularization subsequent to transplantation, neovascularization subsequent to retinal detachment or retinal degeneration, neovascular glaucoma, anterior chamber and/or anterior chamber angle neovascularization, choroidal neovascularization (CNV), subretinal neovascularization, retrolental fibroplasias, ocular histoplasmosis syndrome, myopic degeneration, angioid streaks, uveitis, rubeosis, retrolental fibroplasias, ocular histoplasmosis, and idiopathic central serous chorioretinopathy, amyotrophic lateral sclerosis, sarcoidosis, scleroderma, lupus, Parkinson's disease, sclerosis, Stevens-Johnson syndrome, neoplasia, Von Willebrand disease, vasculitis, and Kawasaki disease.

The phrases “co-administering” or “administering in combination” as used herein mean that two (or more) agents are administered in temporal juxtaposition. The co-administration or combination may be effected by the two agents being mixed into a single formulation, or by the two agents being administered separately but simultaneously, or separately and within a short time of each other. For example, in general the two agents are co-administered within the time range of 6-168 hours. In this case, the agents may be administered in either order, i.e. the chemotherapeutic drug may be administered first, or the one or more oxathiazin-like compounds of the present disclosure may be administered first. In some aspects, the two agents are co-administered in a single formulation, or are co-administered sequentially and separately.

In some aspects, the patient suffers from cancers or tumors including, but not limited to biliary tract cancer; brain cancer, including glioblastomas and medulloblastomas; breast cancer; triple negative breast cancer; uterine cancer; tubal cancer; cervical cancer; choriocarcinoma; colon cancer; bladder cancer; endometrial cancer; retinoblastoma; vaginal cancer; vulvar cancer; esophageal cancer; mouth cancer; gastric cancer; kidney cancer; hematological neoplasms, including acute lymphocytic and myelogenous leukemia; multiple myeloma; AIDS-associated leukemias and adult T-cell leukemia lymphoma; intraepithelial neoplasms, including Bowen's disease and Paget's disease; liver cancer (hepatocarcinoma); lung cancer; head or neck cancers or oral cancers (mouth, throat, esophageal, nasopharyngeal, jaw, tonsil, nasal, lip, salivary gland, tongue, etc.); lymphomas, including Hodgkin's disease and lymphocytic lymphomas; neuroblastomas; neuroendocrine tumors; oral cancer, including squamous cell carcinoma; adrenal cancer; anal cancer; angiosarcoma; appendix cancer; bile duct cancer; bone cancer; carcinoid tumors; soft tissue sarcoma; rhabdomyosarcoma; eye cancer; ovarian cancer, including those arising from epithelial cells, stromal cells, germ cells and mesenchymal cells, and fallopian tube cancer; gallbladder cancer; pancreatic cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma [teratomas, choriocarcinomas]), stromal tumors and germ cell tumors; penile cancer; hemangioendothelioma; gastrointestinal cancer; ureteral cancer; urethral cancer; spinal cancer; pituitary gland cancer; primary central nervous system (CNS) lymphoma; thyroid cancer, including thyroid adenocarcinoma and medullar carcinoma; and renal cancer including adenocarcinoma and Wilms tumor. In some aspects, cancers or tumors include breast cancer, prostate cancer, colorectal cancer, lymphoma, multiple myeloma, and melanoma.

In some aspects, the cancer is a Homologous Recombination Deficiency (HRD) ovarian cancer. In some aspects, the cancer is a Homologous Recombination Proficiency (HRP) ovarian cancer. In some aspects, the ascites is ovarian cancer-associated ascites. In some aspects, the ascites volume is reduced by at least 1.5-, 2-, 3-, 4-, 5-, 6-, 7-, 8-, 9-, 10-, 15-, 20-, 30-, 40-, 50-, 60-, 70-, 80-, or 90-fold when using the inventive combination compared to control. In some aspects, the ascites volume is reduced by at least 1.5-, 2-, 3-, 4-, 5-, 6-, 7-, 8-, 9-, 10-, 15-, 20-, 30-, 40-, 50-, 60-, 70-, 80-, or 90-fold when using the inventive combination compared to GP-2250alone. In some aspects, the ascites volume is reduced by at least 1.5-, 2-, 3-, 4-, 5-, 6-, 7-, 8-, 9-, 10-, 15-, 20-, 30-, 40-, 50-, 60-, 70-, 80-, or 90-fold when using the inventive combination compared to an anti-VEGF drug alone.

Toxicity and therapeutic efficacy of such molecules can be determined by standard pharmaceutical procedures in cell cultures or experimental animals, e.g., by determining the LD50 (the dose lethal to 50% of the population) and the ED50 (the dose therapeutically effective in 50% of the population). The dose ratio of toxic to therapeutic effects is the therapeutic index, which can be expressed as the ratio LD₅₀/ED₅₀.

As used herein, the term “therapeutic index” with regard to a chemotherapeutic drug indicates safety of the chemotherapeutic drug. In some aspects, the therapeutic index can include a comparison of the amount of a therapeutic agent that causes the therapeutic effect (e.g., killing cancer cells) to the amount of the therapeutic agent that causes toxicity (e.g., liver toxicity). It is contemplated that according to certain embodiments an improved therapeutic index can occur using the compositions and/or methods described herein, including without limitation when: (1) the dosage of chemotherapeutic drug is increased above the current therapeutic dosages; (2) the dosage of chemotherapeutic drug remains the same as the current therapeutic dosages; or (3) the dosage of chemotherapeutic drug is decreased below the current therapeutic dosages. In some embodiments, the compositions and methods, including the scenarios in this paragraph can elicit improved or similar therapeutic effect as seen with the current therapeutic dosages with no worse, fewer, or no toxicities.

In one aspect, the present disclosure includes a method of inhibiting or reducing endothelial sprouting by administering one or more compounds of the present disclosure to a subject in need thereof by administering one or more oxathiazin-like compounds to a subject.

In one aspect, the present disclosure includes a method of inhibiting or reducing endothelial sprout length by administering an anti-VEGF drug and one or more oxathiazin-like compounds of the present disclosure to a subject in need thereof.

In one aspect, the present disclosure includes a method of inhibiting or reducing VEGF secretion in cancer cells of a subject by administering an anti-VEGF drug and one or more oxathiazin-like compounds of the present disclosure to the subject.

In one aspect, the present disclosure includes a method of inhibiting or reducing HIF1α expression in cancer cells of a subject by administering an anti-VEGF drug and one or more oxathiazin-like compounds of the present disclosure to the subject.

In one aspect, the present disclosure includes a method of inhibiting or reducing tumor weight in a tumor of a subject by administering an anti-VEGF drug and one or more oxathiazin-like compounds of the present disclosure to the subject.

In one aspect, the present disclosure includes a method of inhibiting or reducing the number of tumor nodules in a subject by administering an anti-VEGF drug and one or more oxathiazin-like compounds of the present disclosure to the subject.

In one aspect, the patient is treated with one or more oxathiazin-like compounds, or a combination thereof, administered intravenously, orally or a combination thereof. In one aspect, the patient is treated with 2250 (also referred to as “compound 2250”, “C-2250”, or “GP-2250”) administered intravenously, orally or a combination thereof.

In some aspects, the present disclosure includes a method further comprising administering the anti-VEGF drug and the compound of formula I in combination with 5-fluorouracil (5-FU)/leucovorin (LV)/irinotecan (IFL), 5-FU/LV/oxaliplatin (FOLFOX4), fluoropyrimidine, irinotecan, oxaliplatin, paclitaxel, PLD (pegylated liposomal doxorubicin), topotecan, cisplatin, Cisplatin/paclitaxel, topotecan/paclitaxel, carboplatin, carboplatin/paclitaxel (PC), gemcitabine, carboplatin/gemcitabine, interferon alfa (IFN), or a combination thereof.

Compounds according to the invention can be administered by any suitable method. Solid dosage forms for oral administration include capsules, tablets, pills, powders, orally-disintegrating tablets, and granules. In such solid dosage forms, the provided composition is mixed with at least one inert, pharmaceutically acceptable excipient and/or fillers or extenders (e.g., starches, lactose, sucrose, glucose, mannitol, and silicic acid), binders (e.g., carboxymethylcellulose, alginates, gelatin, polyvinylpyrrolidinone, sucrose, and acacia), humectants (e.g., glycerol), disintegrating agents (e.g., agar, calcium carbonate, potato starch, tapioca starch, alginic acid, certain silicates, and sodium carbonate), solution retarding agents (e.g., paraffin), absorption accelerators (e.g., quaternary ammonium compounds), wetting agents (e.g., cetyl alcohol and glycerol monostearate), absorbents (e.g., kaolin and bentonite clay), and lubricants (e.g., talc, calcium stearate, magnesium stearate, solid polyethylene glycols, sodium lauryl sulfate), and mixtures thereof. In the case of capsules, tablets and pills, the dosage form may comprise buffering agents.

Solid compositions of a similar type may be employed as fillers in soft and/or hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally comprise opacifying agents and can be of a composition that they release the provided composition(s) only in, or targeting, a certain part of the intestinal tract, optionally, in a delayed manner. Examples of embedding compositions which can be used include polymeric substances and waxes. Solid compositions of a similar type may be employed as fillers in soft and hard-filled gelatin capsules using such excipients as lactose or milk sugar as well as high molecular weight polyethylene glycols and the like.

In certain aspects, capsules may contain an excipient formulation containing one or more of hydroxypropyl methylcellulose (HPMC), gelatin, meglumine, and fish gelatin. In certain aspects, a capsule may contain compound 2250 in combination with taurolidine and/or taurultam. The capsule may optionally further contain one or more of lycopene, ellagic acid (polyphenol), curcumin, piperine, delphinidin, resveratrol, isothiocyanates such as sulforaphane, capsaicin, and piperlongumine.

When used in the form of microparticles or nanoparticles, the compounds of the claimed invention may achieve higher blood levels. The present invention includes microparticles and/or nanoparticles of the compounds of the present disclosure in tablet form or encapsulated in capsules.

In certain aspects, this disclosure relates to administering an oxathiazin-like compound orally to a patient. In some aspects, an oxathiazin-like compound is formulated in capsules or tablets. In certain aspects, oral dosage forms contain between about 50-1000 mg of an oxathiazin-like compound. In certain aspects, oral dosage forms contain between about 100-500 mg of an oxathiazin-like compound. In certain aspects, oral dosage forms contain between about 200-400 mg of an oxathiazin-like compound. In certain aspects, oral dosage forms contain between about 250-350 mg of an oxathiazin-like compound. In certain aspects, the oxathiazin-like compound is C-2250.

In some aspects, the oxathiazin-like compound is provided in a composition at a concentration of about 0.01 to about 1000 μg/ml. In some embodiments, the compounds are administered in compositions at a concentration of about 1 to about 100 μg/ml. In some embodiments, the compounds are administered in compositions at a concentration of about 10 to about 50 ng/ml.

In some aspects, the oxathiazin-like compound is provided in a composition at a concentration of about 0.001 to about 5 wt. %, about 0.01 to about 3.5 wt. %, about 0.1 to about 3 wt. %, about 0.5 to about 2.5 wt. %, or about 1 to about 2 wt. %. In some aspects, the oxathiazin-like compound is provided in a composition at a concentration of about 0.01 to about 1.5%. In some aspects, the oxathiazin-like compound is provided in a composition at a concentration of about 0.1% to about 1%. In some aspects, the oxathiazin-like compound is provided in a composition at a concentration of about 100 to about 5000 μM, about 250 to about 2500 μM, about 500 to about 2000 μM, about 750 to about 1500 μM, about 1000 to about 1250 μM, or any other concentration within the recited ranges.

In some aspects, the oxathiazin-like compound is provided in a composition in a unit dosage form. As used herein, a “unit dosage form” is a composition containing an amount of oxathiazin-like compound that is suitable for administration to an animal, such as a mammal, e.g., a human subject, in a single dose, according to a good medical practice. These compositions may contain from about 0.1 mg (milligrams) to about 500 mg, for example from about 5 mg to about 350 mg of oxathiazin-like compound. The frequency of treatment with the composition of the invention may be changed to achieve and maintain the desired target plasma level. Thus, non-limiting examples of treatment schedules include daily, twice daily, three times daily, weekly, biweekly, monthly, and combinations thereof. Alternatively, the composition of the invention may also be administered as a continuous infusion or a bolus following by one, two, three or more different continuous infusions, e.g., at different rates and dosages of administered drug, such regimens optionally interrupted by one or more additional bolus injections.

In one aspect, the one or more compounds of the present disclosure are provided in a composition that is administered to a subject in need thereof at a total daily dosage may be about 0.001 g to about 1000 g, e.g., about 0.01 g to about 500 g, 0.1 to 300 g, 0.5 to 200 g, 1 g to 100 g, or any amount within the recited range. The daily dosage may be administered in the form of an orally administrable composition. The daily dosage may be administered in the form of a capsule, a tablet, or a pharmaceutically acceptable solution. The daily dosage may be administered in a form that contains one or more compounds of the present disclosure at a concentration of about 0.01 to about 5% w/v, about 0.1 to about 3% w/v, about 0.5 to about 2.5% w/v, or about 1 to about 2% w/v.

The daily dosage may be administered in a form that contains one or more compounds of the present disclosure at a concentration of about 0.001 μg/ml to about 1000 μg/ml, about 0.01 μg/ml to about 750 μg/ml, about 0.05 μg/ml to about 500 μg/ml, about 0.1 μg/ml to about 300 μg/ml, about 0.5 μg/ml to about 200 μg/ml, about 1 μg/ml to about 100 μg/ml, about 5 μg/ml to about 50 μg/ml, about 10 μg/ml to about 25 μg/ml, or about 15 μg/ml to about 20 μg/ml. The daily dosage may be administered in a form that contains one or more solubilizing agents, e.g., polyols.

Effective dosage amounts provided in a composition may include dosage units containing about 0.01-500 mg/kg, about 1-100 mg/kg per day, or about 5-50 mg/kg per day of the e or more compounds of the present disclosure. In some aspects, dosage units are administered every other day, biweekly, or weekly.

In one embodiment, the compound of Formula I is administered to the subject at a total daily dose of from about 0.1 g to about 100 g, about 1 g to about 80 g, about 2 g to about 50 g, or about 5 g to about 30 g.

Suitable formulations for injection or infusion may comprise an isotonic solution containing one or more solubilizing agents, e.g., sugars, polyols, surfactants, osmoticants, in order to provide solutions of increased compound concentration. Such solutions are described in EP 253662B1. The solution can be rendered isotonic with ringer solution or ringer lactate solution. The concentration of the compound in such solutions may be in the range 1-60 g/liter.

The term “polyol” as used herein refers to sugars that contains many hydroxyl (—OH) groups compared to a normal saccharide. Polyols include alcohols and carbohydrates such as mannitol, sorbitol, maltitol, xylitol, isomalt, erythritol, lactitol, sucrose, glucose, galactose, fructose, fucose, ribose, lactose, maltose and cellubiose.

In certain embodiments, the invention also relates to derivatives of the above compounds having, e.g., at least one activity as described herein of said compounds, for example, at least 25%, 30%, 40%, 50%, 60%, 70%, 80%, 90%, 95%, 100%, or more, of said activity.

In certain embodiments, the invention also relates to compositions containing the compounds described herein, including pharmaceutically acceptable solutions of said compounds, as well as orally administrable compositions such as capsules and tablets containing said compositions.

In certain embodiments, the compounds of the present invention can be administered to a subject or patient by any suitable means, for example, in solution, e.g., locally, systemically such as by intravenous infusion, or the like.

Effective dosage amounts of the compounds are dosage units within the range of about 0.1-1,000 mg/kg, preferably 150-450 mg/kg per day, and most preferably 300-450 mg/kg per day.

The specific effective dose for any particular patient will depend on a variety of factors including the severity or likelihood of the neovascularization and/or excessive angiogenesis, disorder or disease; activity of the specific compound employed; the age, body weight, general health, sex and diet of the patient; the preparation of the specific compound; the time and route of administration; the duration of administration; therapeutic agents used in combination or coinciding with the specific compound employed; and like factors known in the medical arts. The effective dose may also change over time as the disorders, diseases, or conditions worsen or improve. For chronic conditions, subjects may receive effective doses for a plurality of days, weeks, months, years, or for the subject's lifetime. The number of and frequency of administrations or co-administrations may vary depending upon the likelihood or severity of the disorder, disease or condition, and the patient specific response to the particular compound administered and/or a second therapeutically active agent that is administered to the subject.

### EXAMPLES

Aspects of the present disclosure will be further described with reference to the following Examples, which are provided for illustrative purposes only and should not be used to limit the scope of or construe the invention.

### Example 1

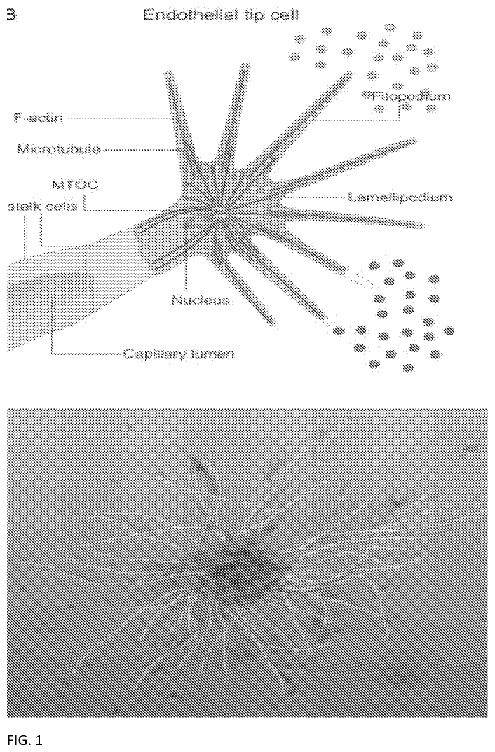

A spheriod assay was performed including growing human endothelial cell (hEC) spheroids and adding VEGF (1000 cells/spheroid; 200 sph/condition) for 24 hours using a hanging drog procedure with methyl-cellulose, then harvesting, adding GP-2250, and suspending in fibrinogen/thrombin matrix. The suspension was then transferred to a 24 well plate and incubated for 24 hours at 37° C. FIG. 1: VEGF-induced Endothelial Sprouting: Impact of GP-2250, Spheroid assay. FIG. 1 shows a schematic endothelial tip cell (top). Tip cells form sprouts, and stalk cells form capillary lumen as shown in the image (bottom). The image (15 spheroids/condition) shows the number and length of sprouts.

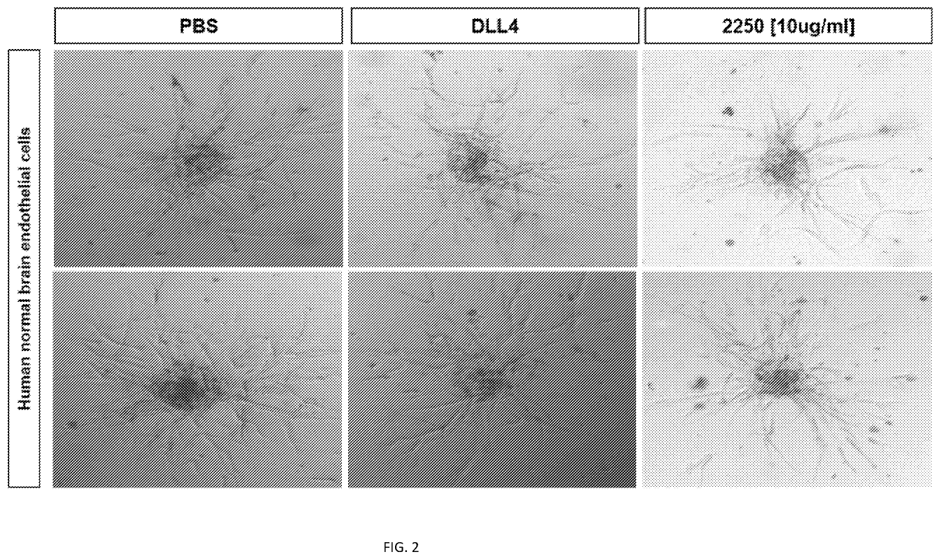

FIG. 2 shows sprouting of endothelial spheroids in human normal brain endothelial cells treated with PBS control, Delta-like 4 (DLL4), and GP-2250 (10 μg/ml).

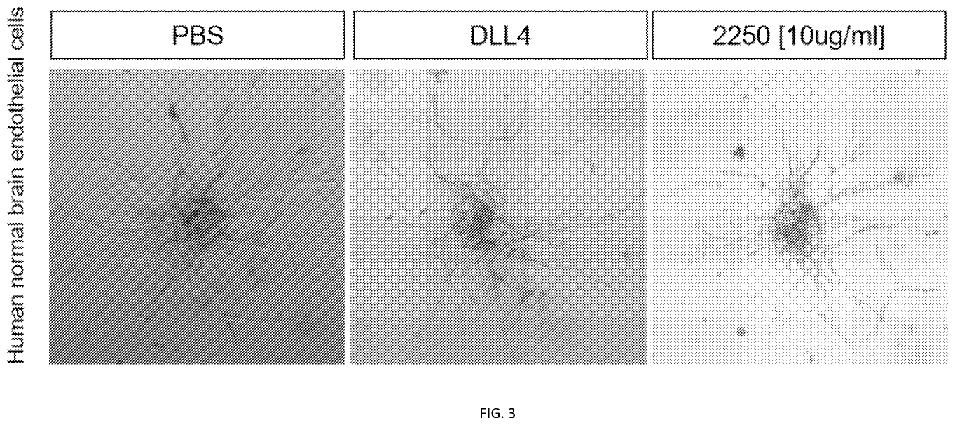

FIG. 3 shows the effects of PBS control, Delta-like 4 (DLL4), and GP-2250 (10 μg/ml) on VEGF-induced endothelial cell sprouting. GP-2250 inhibits VEGF-induced endothelial cell sprouting.

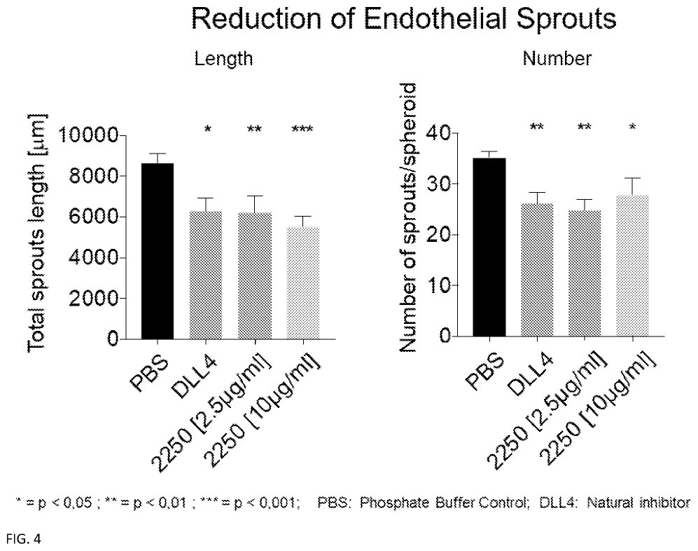

FIG. 4 quantifies the effects of PBS control, Delta-like 4 (DLL4), and GP-2250 (at 2.5 μg/ml or at 10 μg/ml) on VEGF-induced endothelial cell sprouting. The total sprout length and number of sprouts per spheroid were measured as shown in FIG. 4 and a statistically significant reductions in lengths and numbers of sprouts were achieved compared to control.

### Example 2

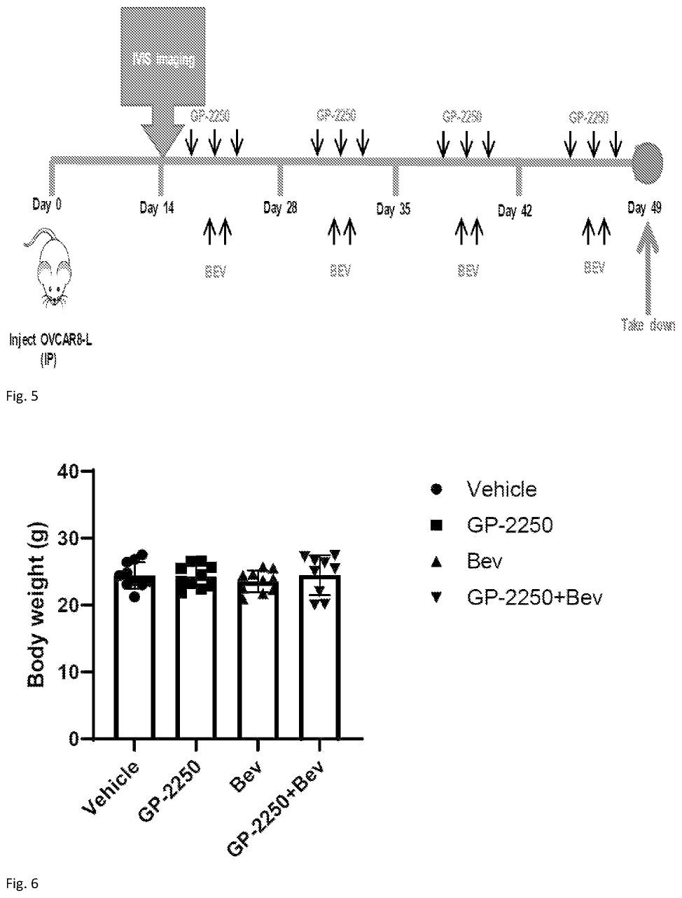

FIG. 5 schematically illustrates the experimental design for in vivo testing of the GP-2250 and bevacizumab on OVCAR8-L mice. The experiment includes testing: 1) IgG control (10 mice, 6.25 mg/kg, BW); 2) GP-2250 (10 mice, 500 mg/kg. 3 times per week); 3) Bevacizumab (10 mice, 6.25 mg/kg, BW) (“BEV”); and 4) the combination of GP-2250 and Bevacizumab.

Tumorigenicity of OVCAR8-L was determined using IVIS imaging. IVIS imaging following OCVAR8-L injection. 1×10⁶ cells were injected through i.p., and then tumorigenicity was measured. All mice were injected with high-grade ovarian cancer cells (OVCAR8) labeled with luciferase gene by i.p. route. All mice survived until the end of the study. No mice were euthanized due to the effect of tumor. Five weeks after treatment, all mice were euthanized, body weight was measured, and no significant body weight change in each group.

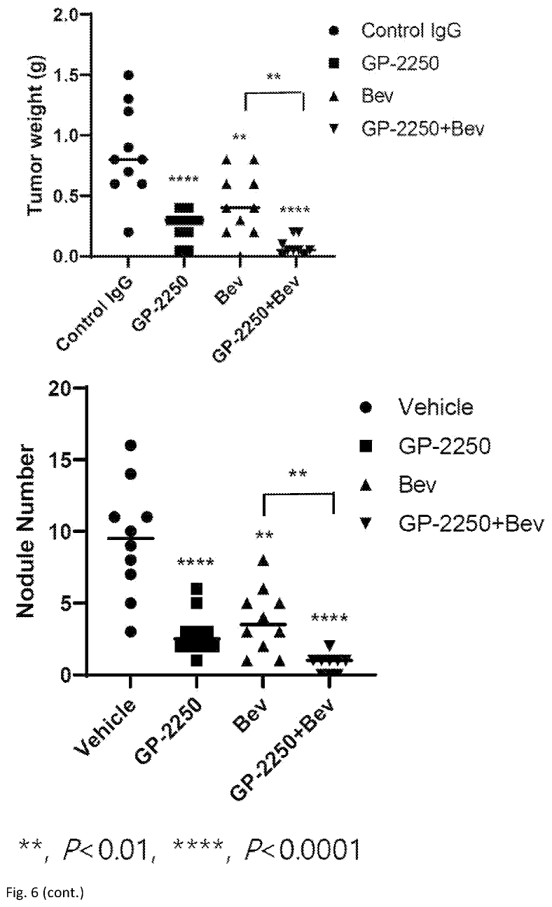

FIG. 6 shows the body weight, tumor weight, and tumor nodule numbers of mice at take down (day 49) with mice in the control and treatment groups. A statistically significant reduction in both tumor weight and tumor nodule number was achieved when using the combination of GP-2250 and bevacizumab compared to using bevacizumab alone as well as compared to control.

### Example 3

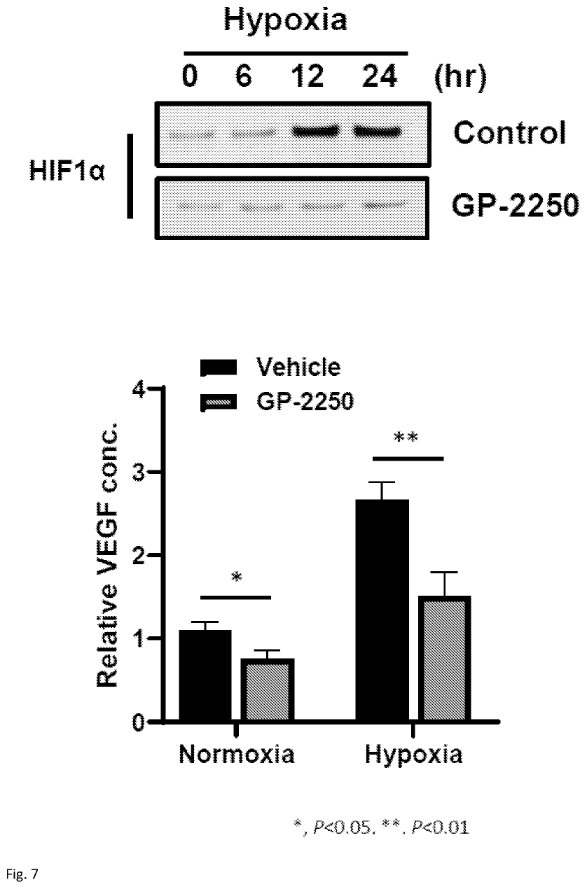

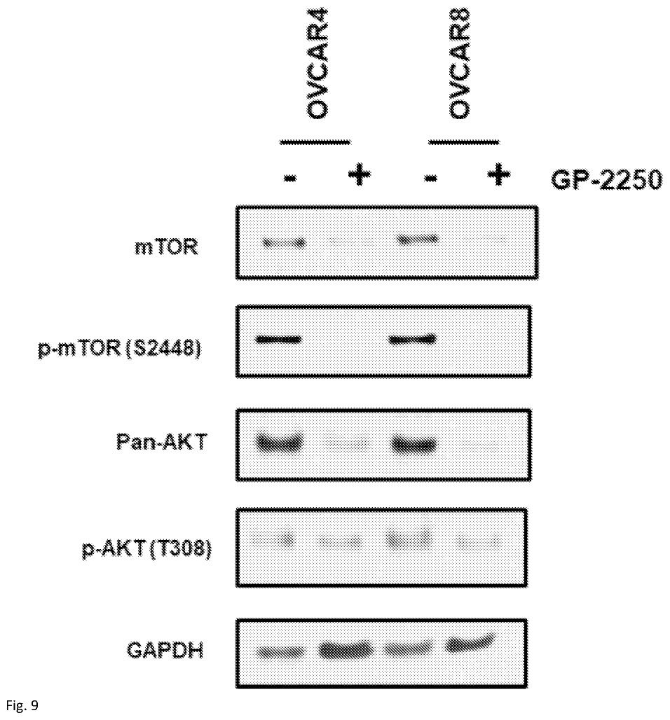

FIG. 7 shows the effect of GP-2250 on HIF1α and VEGF expression. GP-2250 significantly reduced VEGF secretion in cancer cells compared to vehicle. GP-2250 reduced HIF1α expression in cancer cells. GP-2250 inhibits mTOR, AKT and HIF-1α expression. FIG. 9 shows Western blots of ovarian cancer cells treated with GP-2250 for 24 hours following Western blotting.

### Example 4

Human Ovarian cancer cell lines: A2780, Coav3, HeyA8, HeyA8-MDR, Kuramochi, OVCAR3, OVCAR5, OVCAR8, and SKOV3 were obtained and used in studies.

In vitro assays: Cell viability assay, Western blotting, Colony formation assay, Reverse-phase protein array, and Ultra-high resolution mass spectrometry analysis were performed.

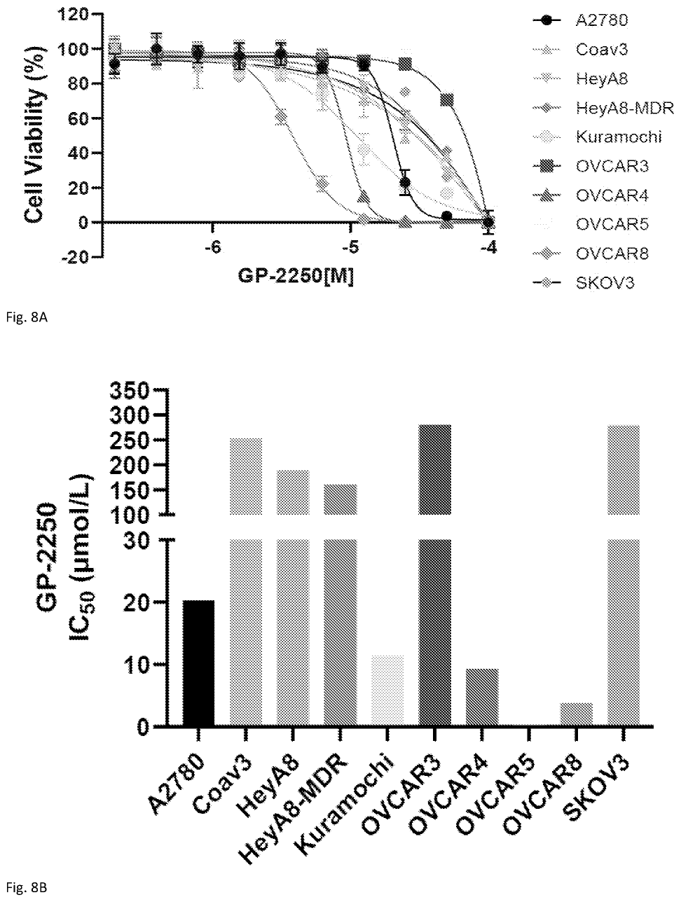

In vivo model of ovarian cancer: Pharmacodynamic study and therapeutic experiment using OVCAR8 ovarian cancer model were performed. FIGS. 8A-8B show the cytotoxic effect of GP-2250 on ovarian cancer cells. FIG. 8A is a cell viability assay and FIG. 8B shows the IC50 of GP-2250.

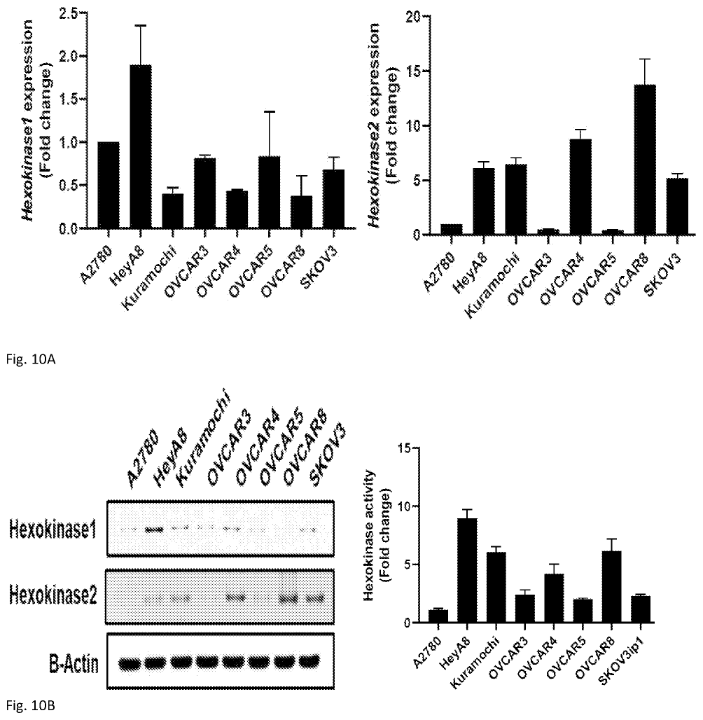

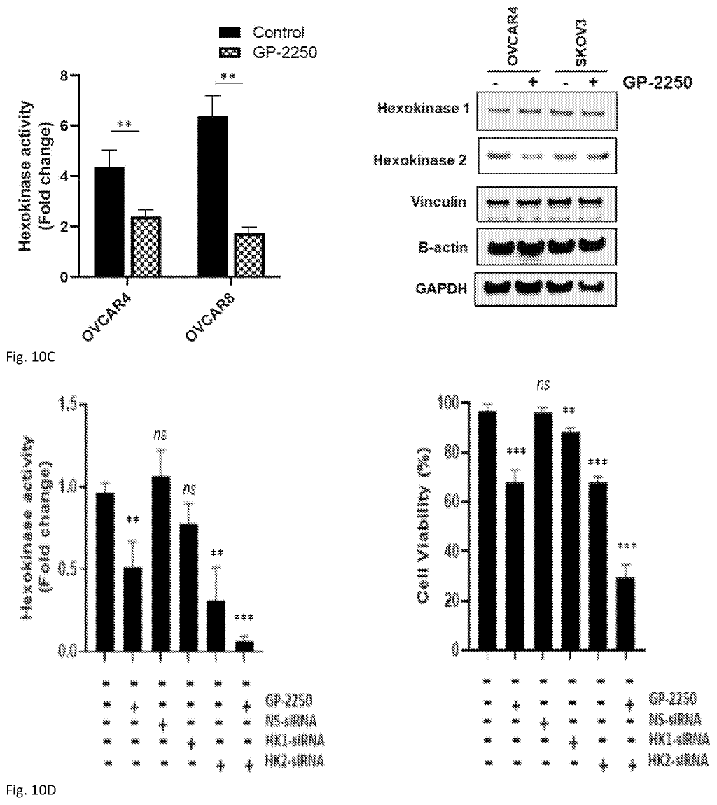

FIGS. 10A-10D show that GP-2250 decreases glycolysis via inhibition of hexokinase 2 activation and expression. FIG. 10A shows mRNA Hexokinase 1 and Hexokinase 2 expression profiles in the various cell lines. FIG. 10B shows the protein and activity of Hexokinase 1 and Hexokinase 2 in the various cell lines. FIG. 10C shows that GP-2250 inhibits hexokinase activity and protein expression levels. FIG. 10D shows siRNA targeting hexokinase 1 and hexokinase 2 that GP-2250 inhibits activity and reduces cancer cell viability. (ns, not significant, **P<0.01; **P<0.001 (vs. control; Student t-test)).

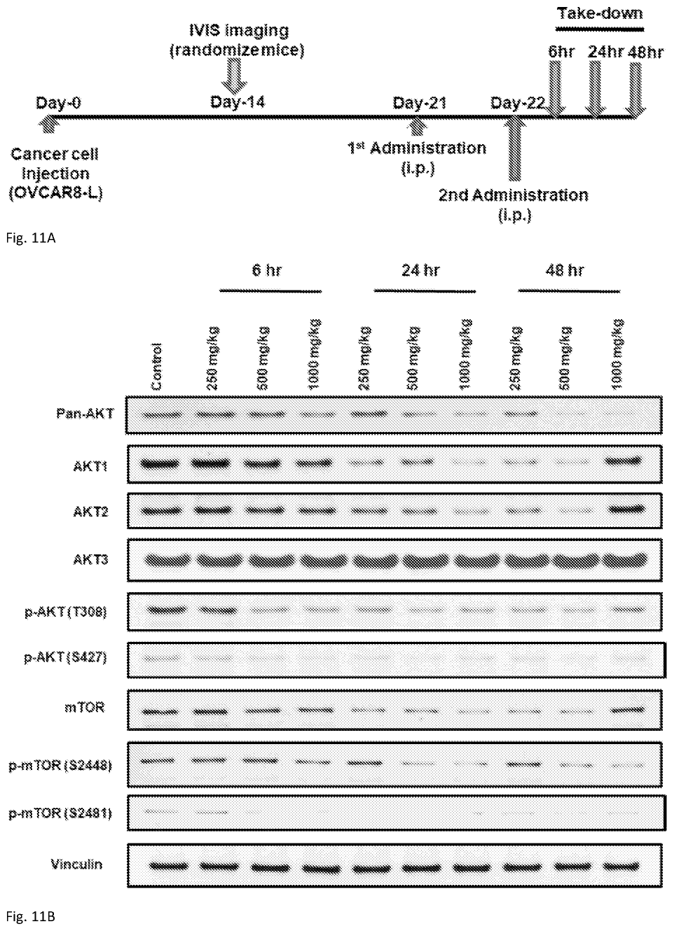

FIGS. 11A-11B show pharmacodynamics studies of GP-2250. FIG. 11A shows a schematic of in vivo PD study of GP-2250. FIG. 11B shows a Western blot and AKT kinase assay of GP-2250.

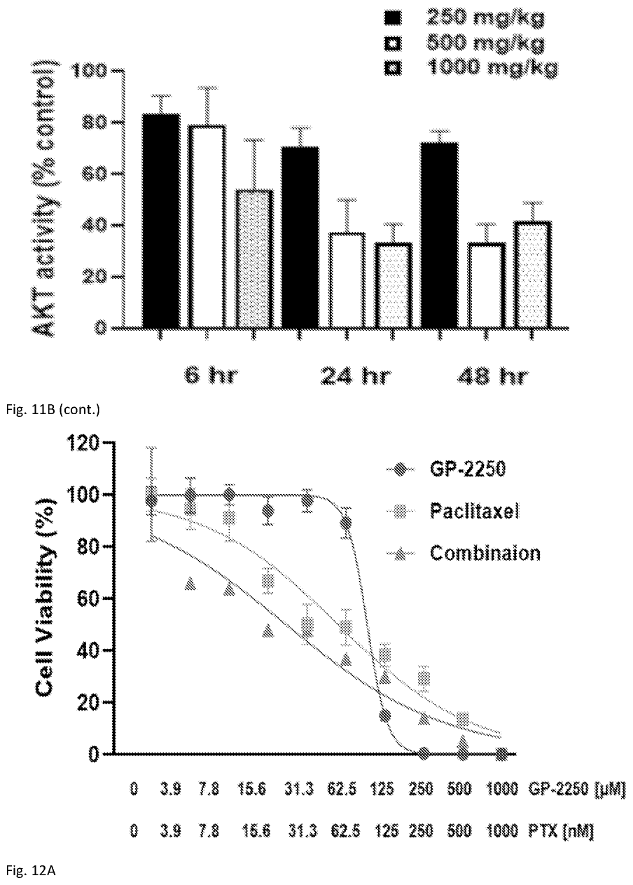

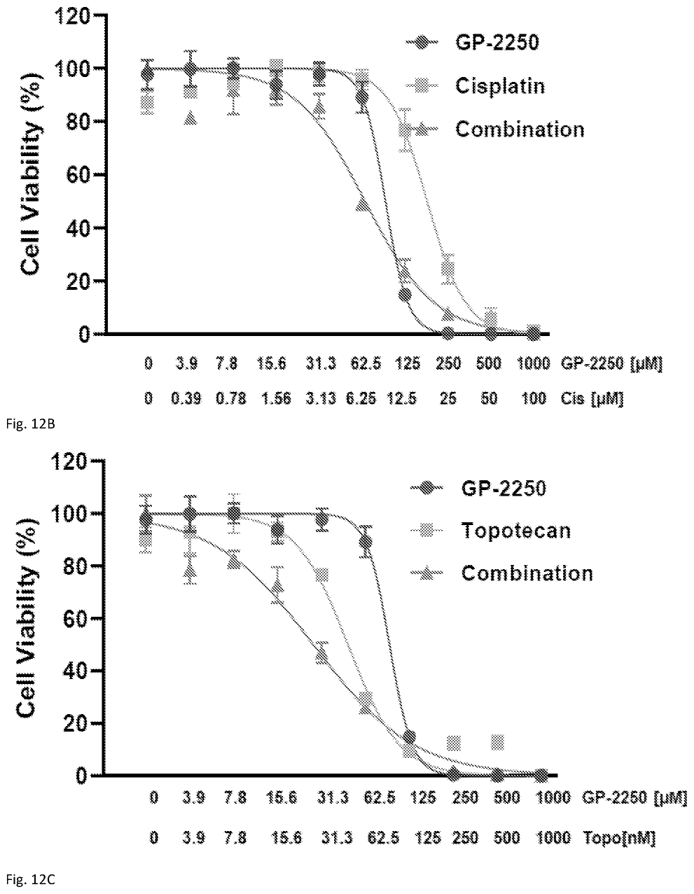

FIGS. 12A-12C show the effect of GP-2250 alone or in combination with other anti-tumor agents. FIG. 12A shows a cell viability assay of GP-2250 alone, paclitaxel alone, or the combination of GP-2250 with paclitaxel. FIG. 12B shows a cell viability assay of GP-2250 alone, cisplatin alone, or the combination of GP-2250 with cisplatin. FIG. 12C shows a cell viability assay of GP-2250 alone, topotecan alone, or the combination of GP-2250 with topotecan.

The cytotoxic effect of GP-2250 in 10 ovarian cancer cell lines and found that Homologous Recombination Deficiency (HRD) ovarian cancer cells (e.g., Kuramochi, OVCAR4, and OVCAR8) were more vulnerable to GP-2250 than Homologous Recombination Proficiency (HRP) ovarian cancer cells (e.g., A2780 and OVCAR5) although both were vulnerable. Thus, GP-2250 reduced proliferation and increased apoptosis in ovarian cancer cells. Reverse Phase Protein Array (RPPA) analyses revealed that GP-2250 inhibited hypoxia-inducible factor-la, AKT, and mTOR activation and expression level. Ultra-high resolution mass spectrometry (HRMS) analysis also revealed that hexokinase 2 activity and expression were significantly reduced by GP-2250 treatment. Furthermore, GP-2250 also reduced glycolysis and ATP synthesis in cancer cells. In vivo pharmacodynamic experiment using the OVCAR8 mouse model demonstrated that a dose of 500 mg/kg GP-2250 was the most effective in downregulating AKT and mTOR activation and expression. In the in vivo therapy experiment using an orthotopic mouse model, a combination of GP-2250 and bevacizumab showed a significant reduction of tumor weights (0.07±0.03 g) and nodules (0.78±0.2) compared to those treated with a vehicle (tumor weight, 0.95±0.1 g and nodules, 8.4±0.65), control IgG groups (tumor weight, 0.86±0.38 and nodules, 9.4±3.92) or the monotherapy groups: GP-2250 (tumor weight, 2.9±0.48 g, and nodules, 2.9±0.48), and bevacizumab (tumor weight, 0.43±0.08 g, and nodules, 3.8±0.71), respectively.

### Example 5

In an ovarian cancer animal model, ascites formation and/or ascites volume is measured using GP-2250 alone, an anti-VEGF drug, e.g., bevacizumab, alone, and the combination of GP-2250 and the anti-VEGF drug. The combination of GP-2250 and the anti-VEGF drug reduces ascites formation and/or volume significantly more than each drug administered alone.

While the subject matter of this disclosure has been described and shown in considerable detail with reference to certain illustrative examples, including various combinations and sub-combinations of features, those skilled in the art will readily appreciate other aspects and variations and modifications thereof as encompassed within the scope of the present disclosure. Moreover, the descriptions of such aspects, combinations, and sub-combinations is not intended to convey that the claimed subject matter requires features or combinations of features other than those expressly recited in the claims. Accordingly, the scope of this disclosure is intended to include all modifications and variations encompassed within the spirit and scope of the following appended claims.

1. A method of treating or delaying progression of cancer in an individual or reducing volume of ascites in the individual or reducing formation of ascites in the individual comprising administering to the individual an effective amount of an anti-VEGF drug and a compound of Formula I: 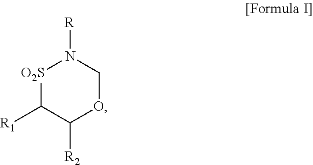
wherein R is H, an in vivo cleavable linker or group, or a leaving group in aqueous solution and R₁ and R₂ are independently, H, alkyl, an aryl, a substituted alkyl, a substituted phenyl, a substituted aryl, or a combination thereof, or a compound selected from the group consisting of the following: 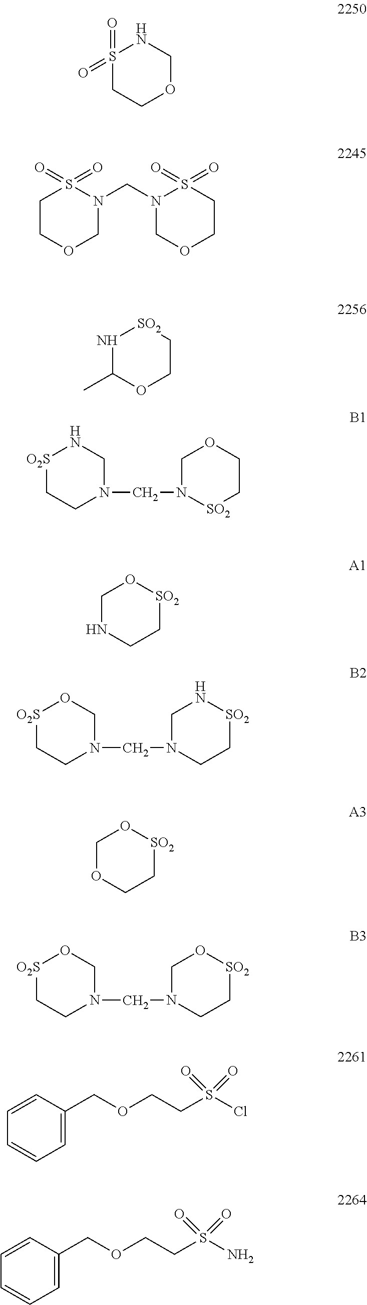 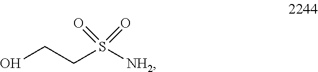
a pharmaceutically acceptable salt, hydrate, ester, prodrug, or solvate thereof.

2. The method of claim 1, wherein the substituted alkyl, substituted phenyl, or substituted aryl may be substituted with one or more halogens or halogen-containing molecules, one or more hydroxyl groups, one or more acyl groups, one or more acyloxy groups, one or more alkoxy groups, one or more aryl groups, one or more carboxy groups, one or more carbonyl groups, one or more alkylcarboxy groups, one or more alkylsufonoxy groups, one or more alkylcarbonyl groups, one or more nitro groups, one or more cyano groups, one or more acylamido groups, one or more phenyl groups, one or more tolyl groups, one or more chlorophenyl groups, one or more alkoxyphenyl groups, one or more halophenyl groups, one or more benzoxazole groups, one or more thiazoline groups, one or more benzimidazole groups, one or more oxazole groups, one or more thiazole groups, one or more indole groups, or a combination thereof.

3. The method of claim 1, wherein the compound of Formula I is compound 2250.

4. The method of any one of claims 1, wherein the anti-VEGF drug is an anti-VEGFR2 antibody; an anti-VEGFR1 antibody; a VEGF-trap; a bispecific VEGF antibody; a bispecific antibody comprising a combination of two arms selected from the group consisting of an anti-VEGF arm, an anti-VEGFR1 arm, and an anti-VEGFR2 arm; an anti-VEGF-A antibody; an anti-VEGFB antibody; an anti-VEGFC antibody; an anti-VEGFD antibody; a nonpeptide small molecule VEGF antagonist; an anti-PDGFR inhibitor; or a native angiogenesis inhibitor.

5. The method of claim 1, wherein the anti-VEGF drug comprises bevacizumab, ramucirumab, tanibirumab, aflibercept, icrucumab, ziv-aflibercept, MP-0250, vanucizumab, sevacizumab, VGX-100, pazopanib, axitinib, vandetanib, stivarga, cabozantinib, lenvatinib, nintedanib, orantinib, telatinib, dovitinig, cediranib, motesanib, sulfatinib, apatinib, foretinib, famitinib, imatinib, tivozanib, or a combination thereof.

6. The method of claim 1, wherein the compound of Formula I is administered in a composition with a pharmaceutically acceptable carrier.

7. The method of claim 1, wherein the compound of Formula I is in the form of an orally administrable composition.

8. The method of claim 7, wherein the composition is in the form of a capsule, a tablet, or a pharmaceutically acceptable solution.

9. The method of claim 6, wherein the compound of Formula I at a concentration of about 0.01 to about 3% w/v.

10. The method of claim 6, wherein the composition comprises the compound of Formula I at a concentration of about 0.01 to about 1000 μg/ml.

11. The method of claim 6, wherein the composition contains one or more solubilizing agents.

12. The method of claim 6, wherein the composition comprises a polyol.

13. The method of claim 6, wherein the composition is an injection and/or infusion formulation comprising a pharmaceutically acceptable injection or infusion carrier.

14. The method of claim 1, wherein the method comprises treating a subject suffering from cancer by administering a combination of a compound of formula I and bevacizumab.

15. The method of claim 1, wherein the method or use comprises treating a subject suffering from cancer by administering a combination of compound 2250 and bevacizumab.

16. The method of claim 1, wherein the cancer is glioblastoma, glioma, neuroblastoma, astrocytoma, carcinomatous meningitis, colon cancer, rectal cancer, colorectal cancer, endometrial cancer, ovarian cancer, breast cancer, prostate cancer, lung cancer, mesothelioma, melanoma, renal cancer, liver cancer, pancreatic cancer, gastric cancer, esophageal cancer, urinary bladder cancer, cervical cancer, cardiac cancer, gall bladder cancer, skin cancer, bone cancer, cancers of the head and neck, leukemia, lymphoma, lymphosarcoma, adenocarcinoma, fibrosarcoma, or a metastasis thereof.

17. The method of claim 1, wherein the cancer is biliary tract cancer; brain cancer, including glioblastomas and medulloblastomas; breast cancer; triple negative breast cancer; uterine cancer; tubal cancer; cervical cancer; choriocarcinoma; colon cancer; bladder cancer; endometrial cancer; retinoblastoma; vaginal cancer; vulvar cancer; esophageal cancer; mouth cancer; gastric cancer; kidney cancer; hematological neoplasms, including acute lymphocytic and myelogenous leukemia; multiple myeloma; AIDS-associated leukemias and adult T-cell leukemia lymphoma; intraepithelial neoplasms, including Bowen's disease and Paget's disease; liver cancer (hepatocarcinoma); lung cancer; head or neck cancers or oral cancers; lymphomas, including Hodgkin's disease and lymphocytic lymphomas; neuroblastomas; neuroendocrine tumors; oral cancer, including squamous cell carcinoma; adrenal cancer; anal cancer; angiosarcoma; appendix cancer; bile duct cancer; bone cancer; carcinoid tumors; soft tissue sarcoma; rhabdomyosarcoma; eye cancer; ovarian cancer, including those arising from epithelial cells, stromal cells, germ cells and mesenchymal cells, and fallopian tube cancer; gallbladder cancer; pancreatic cancer; prostate cancer; rectal cancer; sarcomas, including leiomyosarcoma, rhabdomyosarcoma, liposarcoma, fibrosarcoma and osteosarcoma; skin cancer, including melanoma, Kaposi's sarcoma, basocellular cancer and squamous cell cancer; testicular cancer, including germinal tumors (seminoma, non-seminoma [teratomas, choriocarcinomas]), stromal tumors and germ cell tumors; penile cancer; hemangioendothelioma; gastrointestinal cancer; ureteral cancer; urethral cancer; spinal cancer; pituitary gland cancer; primary central nervous system (CNS) lymphoma; thyroid cancer, including thyroid adenocarcinoma and medullar carcinoma; or renal cancer.

18. The method of claim 1, wherein the cancer is breast cancer, prostate cancer, colorectal cancer, lymphoma, multiple myeloma, or melanoma.

19. The method of claim 1, wherein the cancer is a Homologous Recombination Deficiency (HRD) ovarian cancer.

20. The method of claim 1, wherein the cancer is a Homologous Recombination Proficiency (HRP) ovarian cancer.

21. The method of claim 1, wherein the ascites is ovarian cancer-associated ascites.

22. The method of claim 1, further comprising contacting tumor stem cells in the subject with combination of the anti-VEGF drug and the compound of formula I.

23. The method of claim 1, wherein the method comprises administering a dosage of 0.1-1,000 mg/kg of the compound of formula I in combination with 1 mg/kg to 100 mg/kg of the anti-VEGF drug.

24. The method of claim 1, wherein the method comprises administering a total daily dose of about 0.1 g to about 100 g of a compound of formula I.

25. The method of claim 1, wherein the method comprises administering the anti-VEGF drug at 5-15 mg/kg of the subject's body weight.

26. The method of claim 25, wherein the anti-VEGF drug is administered at 5 mg/kg of the subject's body weight once every 2 or 3 weeks.

27. The method of claim 25, wherein the anti-VEGF drug is administered at 10 mg/kg of the subject's body weight once every 2 or 3 weeks.

28. The method of claim 25, wherein the anti-VEGF drug is administered at 15 mg/kg of the subject's body weight once every 2 or 3 weeks.

29. The method of claim 1, wherein the method comprises administering the anti-VEGF drug at about 20 mg to about 2000 mg of the anti-VEGF drug.

30. The method of claim 1, wherein the method comprises administering the anti-VEGF drug at about 420 mg, about 525 mg, about 840 mg, or about 1050 mg of the anti-VEGF drug.

31. The method of claim 1, wherein the method comprises administering the anti-VEGF drug every week, every 2 weeks, every 3 weeks, or every 4 weeks.

32. The method of claim 24, wherein the anti-VEGF drug is bevacizumab.

33. The method of claim 1, wherein the anti-VEGF drug is administered via oral, intravenous, intraperitoneal, subcutaneous, intramuscular, topical, intradermal, intranasal or intrabronchial administration.

34. The method of claim 1, wherein the anti-VEGF drug is administered via parenteral administration.

35. The method of claim 1, wherein the anti-VEGF drug is administered via intravenous administration.

36. The method of claim 1, further comprising administering the anti-VEGF drug and the compound of formula I in combination with 5-fluorouracil (5-FU)/leucovorin (LV)/irinotecan (IFL), 5-FU/LV/oxaliplatin (FOLFOX4), fluoropyrimidine, irinotecan, oxaliplatin, paclitaxel, PLD (pegylated liposomal doxorubicin), topotecan, cisplatin, Cisplatin/paclitaxel, topotecan/paclitaxel, carboplatin, carboplatin/paclitaxel (PC), gemcitabine, carboplatin/gemcitabine, interferon alfa (IFN). or a combination thereof.
